(19) United States
(12) Patent Application Publication
(10) Pub. No.: US 2023/0382946 A1
(43) Pub. Date: Nov. 30, 2023

(54) POLYPEPTIDE OR PROTEIN DIRECTIONAL MODIFICATION METHOD BASED ON SULFHYDRYL-ALKENYL AZIDE COUPLING

(71) Applicant: Changzhou University, Changzhou (CN)

(72) Inventors: Hua-Dong Xu, Changzhou (CN); Mei-Hua Shen, Changzhou (CN); Yu-Jiao Wang, Changzhou (CN); Yong Wang, Changzhou (CN); Ying Zhou, Changzhou (CN); Xiao-Qian Liu, Changzhou (CN); Jia Guo, Changzhou (CN); Mingxing Ouyang, Changzhou (CN); Linhong Deng, Changzhou (CN)

(21) Appl. No.: 18/322,699

(22) Filed: May 24, 2023

(30) Foreign Application Priority Data
May 26, 2022 (CN) 2022105873969

(51) Int. Cl. C07K 1/10; C07K 1/107; C07K 1/113

(52) U.S. Cl. CPC C07K 1/10; C07K 1/1077; C07K 1/113

(57) ABSTRACT

A polypeptide or protein directional modification method based on sulfhydryl-alkenyl azide coupling is provided. The method uses a sulfhydryl group-containing compound and a compound containing alkenyl azide group as reactants to generate an amino acid containing β-carbonyl sulfide, a polypeptide containing β-carbonyl sulfide and a protein bioconjugate containing β-carbonyl sulfide, thereby achieving a chemical modification. The method is mild in conditions and wide in solvent selectivity, a reaction temperature is in a range of 37 degrees Celsius (° C.) to 40° C., and a reaction time is in a range of 10 minutes to 48 hours. The method is promising in preparing functional polypeptides or functional proteins, protein labeling, and biological medicine.

### TECHNICAL FIELD

The disclosure relates to the field of biotechnology, and particularly to a polypeptide or protein directional modification method based on sulfhydryl-alkenyl azide coupling.

### BACKGROUND

Chemical modification of biomacromolecules refers to mounting groups with specific functions onto target biomacromolecules by means of chemical reactions. Polypeptide or protein is a main target of the chemical modification, because the chemical modification can improve the function of the polypeptide and the protein and can also endow the polypeptide or the protein with a new function, and the chemical modification of the polypeptide or the protein plays an important role in life science, medicine and biological materials.

Cysteine (Cys) is an important and unique amino acid, which contains a sulfhydryl group regarded as a catalytic site for various enzymes. Compared to other nucleophilic groups, the sulfhydryl group exhibits distinct reactivity and can be found in 97% of human proteins, which means that the cysteine-based chemical modification is easy to achieve site selectivity and has universal applicability. The chemical modification of the specific protein cysteine is usually based on the following three reactions (formulas of the three reactions are expressed as follows):

The formula I is an exchange reaction of disulfide, in which a target cysteine and a disulfide reagent are subjected to thiol exchange to transfer the functional group. The method has good specificity and mild conditions, but the new disulfide bond will continue to undergo thiol exchange and lose functional molecules. The formula II is a nucleophilic substitution reaction, in which Cys is alkylated by a functional molecule carrying an electrophilic site but the reaction is susceptible to interference by other nucleophilic residues. The formula III is a Michael addition reaction, in which a Michael acceptor loaded with functional small molecules is added with a sulfhydryl group (also referred to as thiol group) to obtain a coupling product, but such coupling product is susceptible to loss of the functional molecules due to an (retro)-Michael reaction.

Recently, other methods are appeared in the related art, including: 1) a nucleophilic aromatic substitution reaction (S_{N}Ar) of a sulfhydryl (also referred to as thiol) side chain of a cysteine on electrophilic aromatic reagents such as polyfluoroarene, sulfonyl aza-aromatic hydrocarbons; 2) alkylation, alkenylation and alkynylation of cysteine residues by perfluoroalkyl, alkenyl and alkynyl hypervalent iodine reagents; and 3) arylation and boronation of cysteine residues by transition metal organic reagents. However, these methods have not been widely used at present due to their respective limitations and are particularly difficult to implement in a pharmaceutical industry.

In addition, an addition reaction of the sulfhydryl group to ordinary alkenes/alkynes, i.e., Thiol-ene/Thiol-yne reaction expressed by a reaction formula as follows, is used for protein modification, but such reaction requires ultraviolet light, photosensitizer or free radical initiator, such additional reagents or conditions and the defects in the efficiency of the reaction itself severely impair the application prospects of these methods.

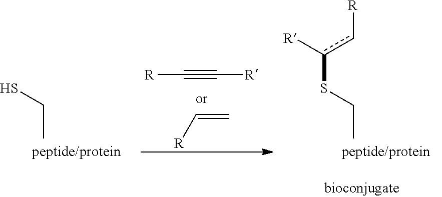

### SUMMARY

In view of the above defects in the related art, the disclosure provides a modification method based on a new mechanism, which can effectively solve the defects in the related art. The disclosure provides a polypeptide or protein directional modification method based on sulfhydryl-alkenyl azide coupling. The chemical principle of the sulfhydryl-alkenyl azide coupling is as follows. A sulfhydryl radical in a sulfhydryl group-containing compound (also referred to as sulfhydryl compound) is added to the β site of a compound containing an alkenyl azide group through free radical Thiol-ene addition (i.e., an addition reaction of the sulfhydryl group to alkenes) to generate an intermediate, the generated intermediate decomposes to release nitrogen molecule, followed by hydrolysis to release ammonia molecule, and then a product is produced, and the product is a compound containing β-carbonyl sulfide. Furthermore, a ketone carbonyl group is chemically and selectively connected to the sulfhydryl group through the above method.

Specifically, the polypeptide or protein directional modification method based on sulfhydryl-alkenyl azide coupling is provided, including: reacting a sulfhydryl group-containing compound with a compound containing alkenyl azide group in a reaction medium to modify the sulfhydryl group-containing compound into a compound containing β-carbonyl sulfide.

A reaction formula is as follows:

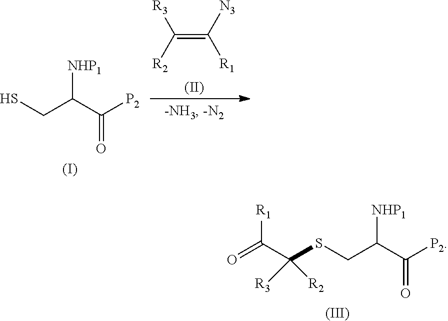

In the reaction formula, the sulfhydryl group-containing compound is one of cysteine and cysteine derivatives with free alkyl sulfhydryl group, which are provided through liquid phase or solid phase synthesis. A general structural formula of the cysteine and the cysteine derivatives is expressed by formula (I) as follows:

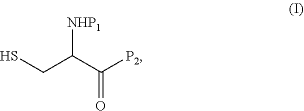

where P₁ in the formula (I) represents one selected from the group consisting of a hydrogen group, an alkyl group, an aryl group, a heteroaryl group, a carbonyl-alkyl group, a carbonyl-aryl group, a carbonyl-heteroaryl group, an ester-alkyl group, an ester-aryl group, an ester-heteroaryl group, a carbonyl-amino-alkyl group, a carbonyl-amino-aryl group, a carbonyl-amino-heteroaryl group, a dipeptidyl group and a polypeptidyl group. The above-mentioned dipeptidyl group and the polypeptidyl group refer to molecular fragments composed of more than two of arbitrary natural amino acids and common unnatural amino acids, or any combination thereof connected by amide bonds; and P₂ in the formula (I) represents one selected from the group consisting of a hydroxyl group, an ether-alkyl group, an amino-alkyl group, an amino-aryl group, an amino-heteroaryl group, an amino-dipeptidyl group, an amino-polypeptidyl group, and a cyclic amino group such as a proline derivative group.

In an embodiment, the sulfhydryl group-containing compound can also be natural polypeptides, natural proteins, genetically engineered polypeptide, and other modified proteins containing the sulfhydryl group. Table 1 illustrates some of amino acids, peptides, and proteins of the disclosure, but the disclosure is not limited to the amino acids, peptides, and proteins collected in the table 1.

TABLE 1
the sulfhydryl group-containing amino acids, peptides, and proteins:

| | |
|---|---|
| 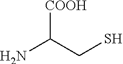 | 25 |
| 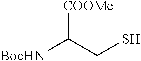 [SMILES: COC(=O)C(CS)NC(=O)OC(C)(C)C] | 26 |
| 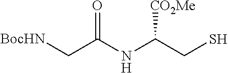 [SMILES: COC(=O)[C@H](CS)NC(=O)CNC(=O)OC(C)(C)C] | 27 |
| 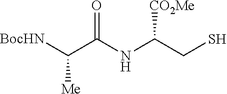 [SMILES: COC(=O)[C@H](CS)NC(=O)[C@H](C)NC(=O)OC(C)(C)C] | 28 |
| 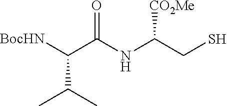 [SMILES: COC(=O)[C@H](CS)NC(=O)[C@@H](NC(=O)OC(C)(C)C)C(C)C] | 29 |
| 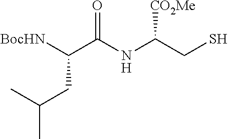 [SMILES: COC(=O)[C@H](CS)NC(=O)[C@H](CC(C)C)NC(=O)OC(C)(C)C] | 30 |
| 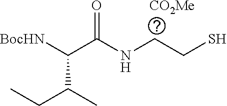 | 31 |
| 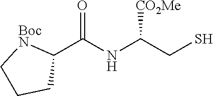 [SMILES: COC(=O)[C@H](CS)NC(=O)[C@@H]1CCCN1C(=O)OC(C)(C)C] | 32 |
| 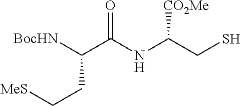 [SMILES: COC(=O)[C@H](CS)NC(=O)[C@H](CCSC)NC(=O)OC(C)(C)C] | 33 |
| 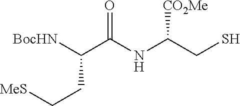 [SMILES: COC(=O)[C@H](CS)NC(=O)[C@H](CCSC)NC(=O)OC(C)(C)C] | 33 |
| 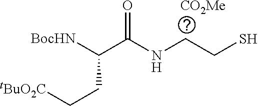 | 34 |
| 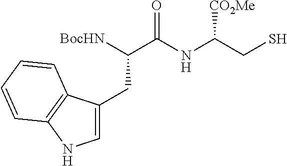 [SMILES: COC(=O)[C@H](CS)NC(=O)[C@H](Cc1c[nH]c2ccccc12)NC(=O)OC(C)(C)C] | 35 |
| 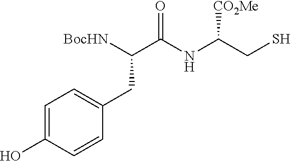 [SMILES: COC(=O)[C@H](CS)NC(=O)[C@H](Cc1ccc(O)cc1)NC(=O)OC(C)(C)C] | 36 |
| 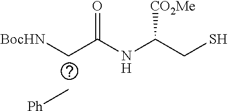 | 37 |
| 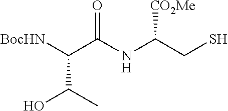 [SMILES: COC(=O)[C@H](CS)NC(=O)[C@@H](NC(=O)OC(C)(C)C)C(C)O] | 38 |
| 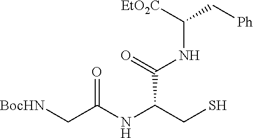 [SMILES: CCOC(=O)[C@H](Cc1ccccc1)NC(=O)[C@H](CS)NC(=O)CNC(=O)OC(C)(C)C] | 39 |
| 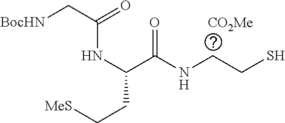 | 40 |
| 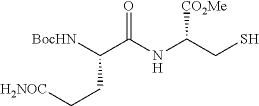 [SMILES: COC(=O)[C@H](CS)NC(=O)[C@H](CCC(N)=O)NC(=O)OC(C)(C)C] | 41 |
| 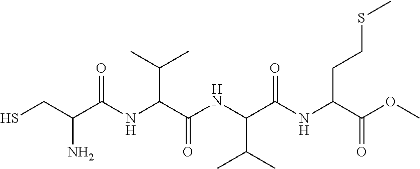 [SMILES: COC(=O)C(CCSC)NC(=O)C(NC(=O)C(NC(=O)C(N)CS)C(C)C)C(C)C] | 42 |
| 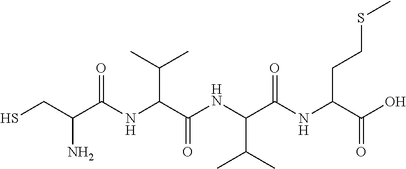 [SMILES: CSCCC(NC(=O)C(NC(=O)C(NC(=O)C(N)CS)C(C)C)C(C)C)C(=O)O] | 43 |
| glutathione (GSH) | 44 |
| 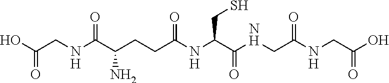 [SMILES: N[C@@H](CCC(=O)N[C@@H](CS)C(=O)NCC(=O)NCC(=O)O)C(=O)NCC(=O)O] | 45 |
| BSA | 46 |
| YFet-ECFP. | 47 |

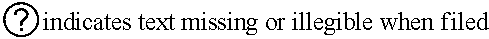

In an embodiment, the sulfhydryl group-containing compound is specifically selected from one of a group consisting of the followings:

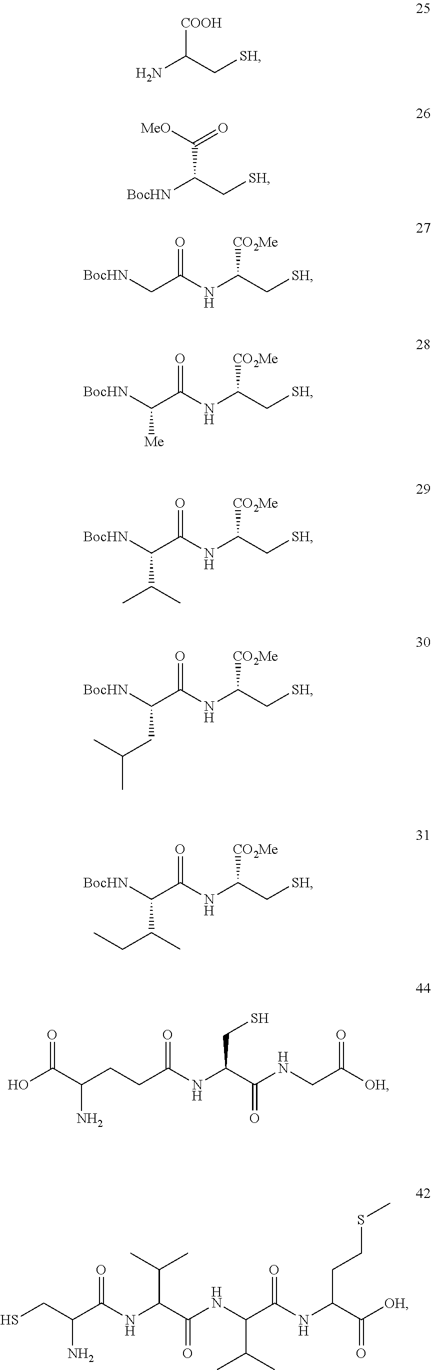

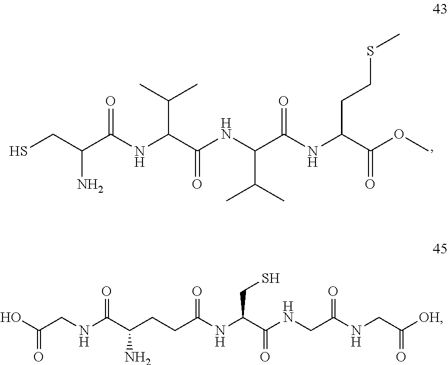 [SMILES: COC(=O)C(CCSC)NC(=O)C(NC(=O)C(NC(=O)C(N)CS)C(C)C)C(C)C ; N[C@@H](CCC(=O)N[C@@H](CS)C(=O)NCC(=O)NCC(=O)O)C(=O)NCC(=O)O]

the bovine serum albumin (BSA) 46, and the modified protein YPet-ECFP 47.

A general structural formula of the compound containing an alkenyl azide group is expressed by formula (II) as follows:

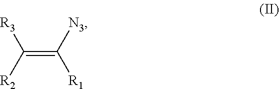

where R₁ is one of an alkyl group and an aryl group, R₂ and R₃ in the formula (II) each are a hydrogen group. Selectively, R₁ is substituted by at least one Q¹ group, and the Q¹ group represents one selected from the group consisting of an amino group, an alkyl group, a cycloalkyl group, a heterocycloalkyl group, an aryl group, a heteroaryl group, a heterocyclic group, an —OR₄ group, a —S(O)ₙR₅ group, a —NR₆R₇ group, a —SO₂NR₆R₇ group, and a —NR₆S(O)₂R₅ group.

The above R₄, R₅, R₆ and R₇ can be substituted by one or more of a hydrogen group, a halogen group, a cyano group (—CN), a hydroxyl group (—OH), an amino group (—NH₂), a nitro group (—NO₂), an oxyl group, a trifluoromethyl group (—CF₃), a trifluoromethoxy group (—O—CF₃), a carboxyl group (—COOH), a —S(O)ₙH group, an alkyl group, an aryl group, a heteroaryl group, a cycloalkyl group, a heterocycloalkyl group, a heterocycloaryl group, and an ether-alkyl group; where n is taken from 0, 1, or 2.

In the formula (II), R₁, R₄, R₅, and Q¹ may also be organic fragments containing functional molecules, such as drug molecules, inhibitors of enzymes, antagonists or agonists of receptors, fluorescent chromonic molecules, (poly) glycosyl, polypeptides, natural product molecules, polydentate ligands, organic catalytic groups.

Table 2 illustrates some of compounds containing the alkenyl azide group according to the disclosure, but the disclosure is not limited to the compounds collected in the table 2.

TABLE 2
some of the compounds containing the alkenyl azide group:

| | |
|---|---|
| 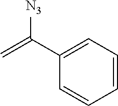 | 1 |
| 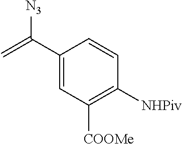 | 2 |
| 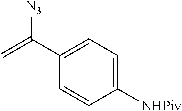 | 3 |
| 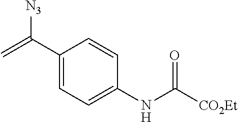 [SMILES: C=C(N=[N+]=[N-])c1ccc(NC(=O)C(=O)OCC)cc1] | 4 |
| 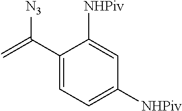 | 5 |
| 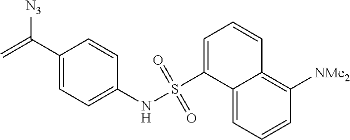 [SMILES: C=C(N=[N+]=[N-])c1ccc(NS(=O)(=O)c2cccc3c(N(C)C)cccc23)cc1] | 6 |
| 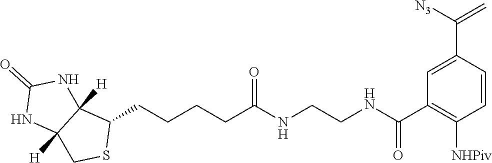 | 7 |
| 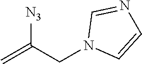 | 8 |
| 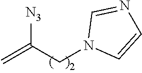 | 9 |
| 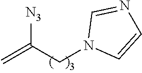 | 10 |
| 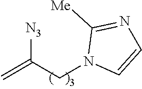 | 11 |
| 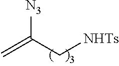 | 12 |
| 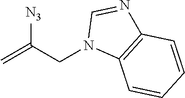 | 13 |
| 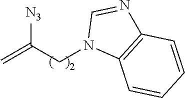 | 14 |
| 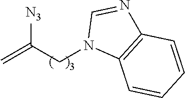 | 15 |
| 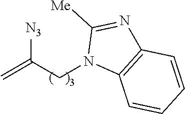 | 16 |
| 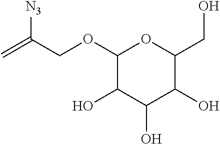 [SMILES: C=C(COC1OC(CO)C(O)C(O)C1O)N=[N+]=[N-]] | 17 |
| 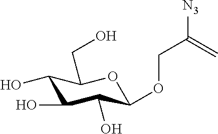 | 18 |
| 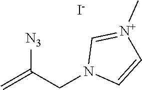 | 19 |
| 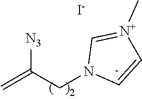 | 20 |
| 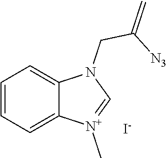 [SMILES: C=C(Cn1c[n+](C)c2ccccc21)N=[N+]=[N-] ; [I-]] | 21 |
| 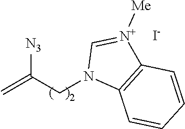 | 22 |
| 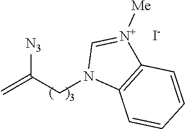 | 23 |
| 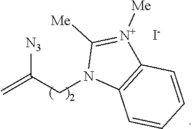 | 24 |

In an embodiment, the compound containing the alkenyl azide group is specifically selected from one of a group consisting of the followings:

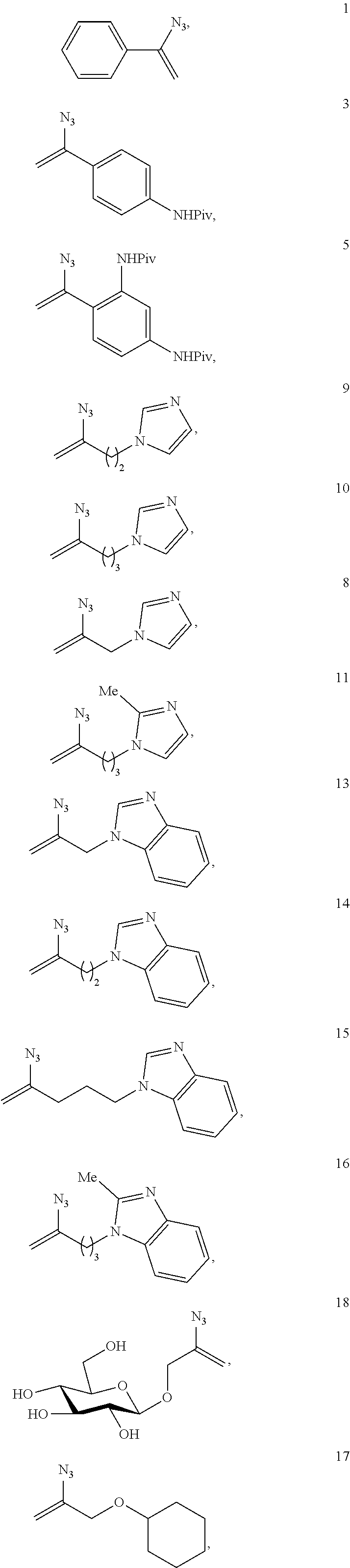

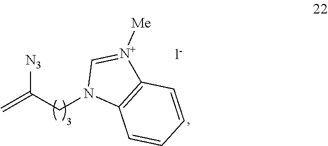

a dansyl-vinyl azide and a streptavidin Alexa Fluor™ 568 conjugate.

A reaction temperature of the above reaction formula is in a range of 0° C. to 60° C., and a reaction time of the above reaction formula is in a range of 10 minutes (min) to 48 hours (h). Specially, the reaction temperature is in a range of 37° C. to 40° C.

The reaction medium is at least one selected from the group consisting of tetrahydrofuran, dioxane, acetone, N,N-dimethylformamide, N-methyl pyrrolidone, dimethyl sulfoxide, water, methanol, ethanol, isopropanol, acetonitrile, and a buffer solution.

The compound containing β-carbonyl sulfide is one selected from the group consisting of a cysteine containing β-carbonyl sulfide, a cysteine derivative containing β-carbonyl sulfide, a polypeptide containing β-carbonyl sulfide and a protein containing β-carbonyl sulfide. A general structural formula of the cysteine containing β-carbonyl sulfide and the cysteine derivative containing β-carbonyl sulfide is expressed by a formula (III) as follows:

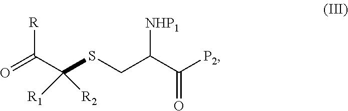

Table 3 illustrates some of modified compounds containing β-carbonyl sulfide, but the disclosure is not limited to the modified compounds in the Table 3.

TABLE 3
some of the modified compounds containing ß-carbonyl sulfide:

| | |
|---|---|
| 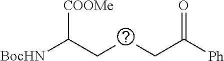 [SMILES: COC(=O)C(CCCC(=O)c1ccccc1)NC(=O)OC(C)(C)C] | 48 |
| 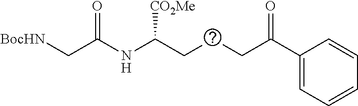 [SMILES: COC(=O)[C@H](CCCC(=O)c1ccccc1)NC(=O)CNC(=O)OC(C)(C)C] | 49 |
| 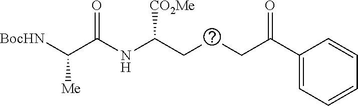 [SMILES: COC(=O)[C@H](CCCC(=O)c1ccccc1)NC(=O)[C@H](C)NC(=O)OC(C)(C)C] | 50 |
| 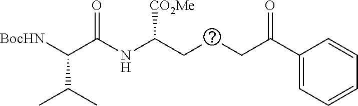 [SMILES: COC(=O)[C@H](CCCC(=O)c1ccccc1)NC(=O)[C@@H](NC(=O)OC(C)(C)C)C(C)C] | 51 |
| 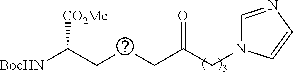 | 52 |
| 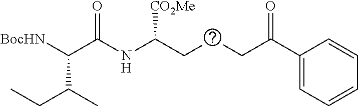 [SMILES: CCC(C)[C@H](NC(=O)OC(C)(C)C)C(=O)N[C@@H](CCCC(=O)c1ccccc1)C(=O)OC] | 53 |
| 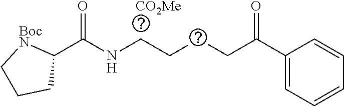 | 54 |
| 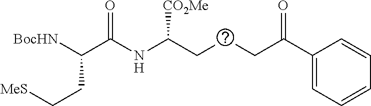 [SMILES: COC(=O)[C@H](CCCC(=O)c1ccccc1)NC(=O)[C@H](CCSC)NC(=O)OC(C)(C)C] | 55 |
| 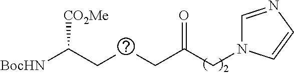 | 56 |
| 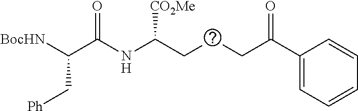 [SMILES: COC(=O)[C@H](CCCC(=O)c1ccccc1)NC(=O)[C@H](Cc1ccccc1)NC(=O)OC(C)(C)C] | 57 |
| 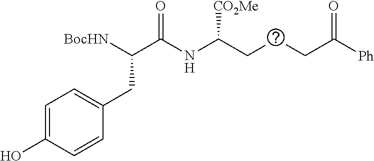 [SMILES: COC(=O)[C@H](CCCC(=O)c1ccccc1)NC(=O)[C@H](Cc1ccc(O)cc1)NC(=O)OC(C)(C)C] | 58 |
| 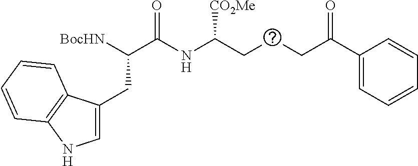 [SMILES: COC(=O)[C@H](CCCC(=O)c1ccccc1)NC(=O)[C@H](Cc1c[nH]c2ccccc12)NC(=O)OC(C)(C)C] | 59 |
| 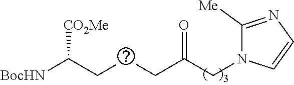 | 60 |
| 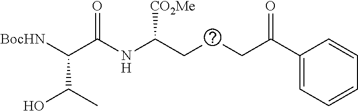 [SMILES: COC(=O)[C@H](CCCC(=O)c1ccccc1)NC(=O)[C@@H](NC(=O)OC(C)(C)C)C(C)O] | 61 |
| 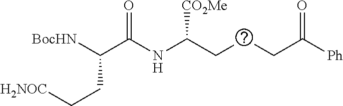 [SMILES: COC(=O)[C@H](CCCC(=O)c1ccccc1)NC(=O)[C@H](CCC(N)=O)NC(=O)OC(C)(C)C] | 62 |
| 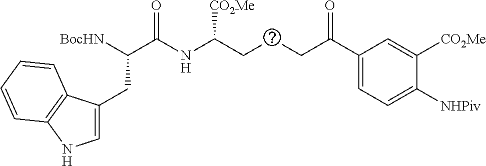 | 63 |
| 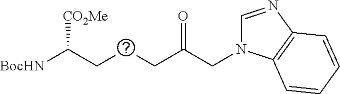 | 64 |
| 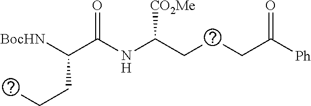 [SMILES: CCC[C@H](NC(=O)OC(C)(C)C)C(=O)N[C@@H](CCCC(=O)c1ccccc1)C(=O)OC] | 65 |
| 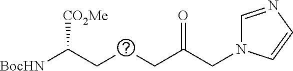 | 66 |
| 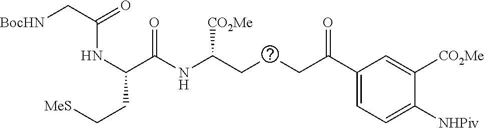 | 67 |
| 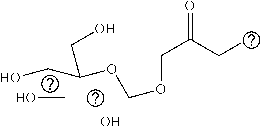 | 68 |
| 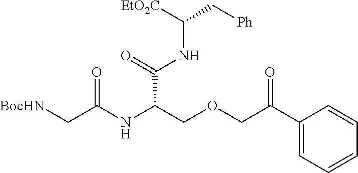 [SMILES: CCOC(=O)[C@H](Cc1ccccc1)NC(=O)[C@H](COCC(=O)c1ccccc1)NC(=O)CNC(=O)OC(C)(C)C] | 69 |
| 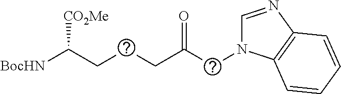 | 70 |
| 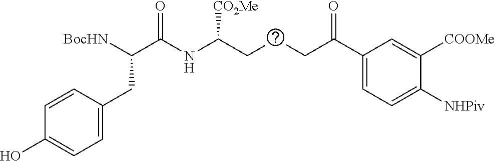 | 71 |
| 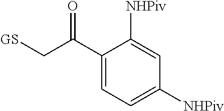 | 72 |
| 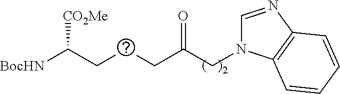 | 73 |
| 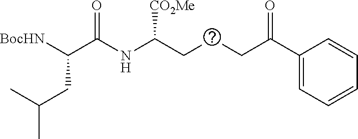 [SMILES: COC(=O)[C@H](CCCC(=O)c1ccccc1)NC(=O)[C@H](CC(C)C)NC(=O)OC(C)(C)C] | 74 |
| 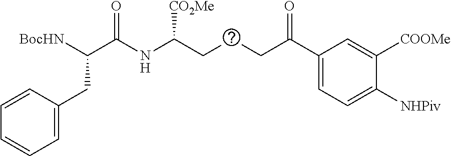 | 75 |
| 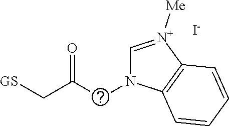 | 76 |
| 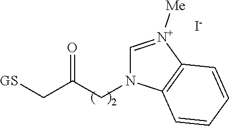 | 77 |
| 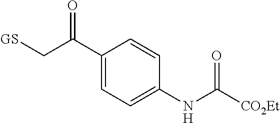 | 78 |
| 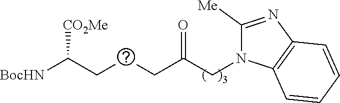 | 79 |
| 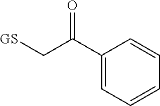 | 80 |
| 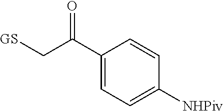 | 81 |
| 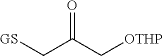 | 82 |
| 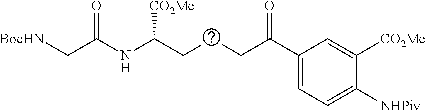 | 83 |
| 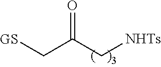 | 84 |
| 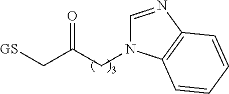 | 85 |
| 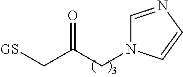 | 86 |
| 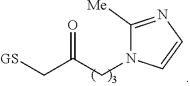 | 87 |

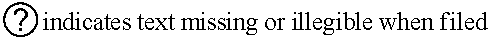

The disclosure may achieve technical effects as follows.
(1) The reaction of the disclosure is a free radical chain reaction initiated by a free radical sulfhydryl group, conditions of the reaction are mild, the reaction is almost not influenced by solvents, and the reaction can be smoothly carried out in organic solvents, water, buffer solvents, and mixed solvents.
(2) The non-electrophilic alkenyl azide used herein is not interfered by other nucleophilic residues, such as an amino group, a (phenol) hydroxyl group, a carboxyl group, an imidazolyl group, an indole, and other groups.
(3) The reaction does not need additives or catalysts, and by-products produced by the reaction are nitrogen and ammonia, which are easy to be post-treated.
(4) The prepared thioether (also referred to as sulfide) is stable, and the ketone carbonyl group can be a convenient secondary modification site.
(5) The obtained compounds are new chemical tools, and the tools facilitate preparing the bioconjugates used in vivo and in vitro, including fluorescent marking of proteins, preparation of antibody-drug conjugates, proteomics analysis, development of covalent inhibitor drugs, etc.

### BRIEF DESCRIPTION OF DRAWINGS

FIG. 1A illustrates an electrophoresis diagram of a sulfonyl-vinyl azide-modified bovine serum albumin (BSA) through Coomassie brilliant blue (CBB) staining.

FIG. 1B illustrates an electrophoresis diagram of the sulfonyl-vinyl azide-modified BSA through fluorescence staining.

FIG. 2 illustrates a schematic diagram of a method for preparing Ni-NTA resin labeled with YPET-ECP and STAV AF568.

FIG. 3 illustrates a fluorescence image of labeled and unlabeled Ni-NTA resin.

### DETAILED DESCRIPTION OF EMBODIMENTS

The disclosure will be further described in combination with embodiments, but not limited to the embodiments.

### Embodiment 1

A cysteine derivative 26 (0.2 millimoles abbreviated as mmol, 1 equivalent abbreviated as equiv) is dissolved in 3 milliliters (mL) of tetrahydrofuran (THF) to obtain a solution. To this solution, alkenyl azide 1 (2 mmol, 10 equiv) is added to obtain a reaction mixture. The mixture is stirred at 25 degrees Celsius (° C.) in an air atmosphere for reacting until polypeptide in the mixture is consumed (monitored by thin-layer chromatography abbreviated as TLC). The reaction completes after 0.5 hour (h), and the solvent is removed under reduced pressure using a rotary evaporator to obtain a crude product. Petroleum ether and ethyl acetate are used as an eluent for the purification of the crude product via column chromatography on silica gel. A volume ratio of the petroleum ether: the ethyl acetate is 3:1 used herein, and then a target product 48 can be rapidly collected after concentration in vacuum, in a yield of 77%. The reaction is expressed by the following formula:

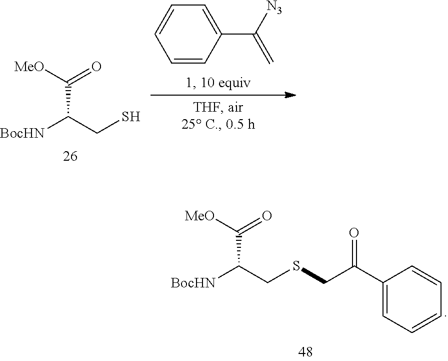

### Embodiment 2

A cysteine derivative 26 (0.2 mmol, 1 equiv) is dissolved in 3 mL of acetonitrile (MeCN) to obtain a solution. To this solution, alkenyl azide 1 (2 mmol, 10 equiv) is added to obtain a reaction mixture. The mixture is stirred at 40° C. under an air atmosphere for reacting until polypeptide in the mixture is consumed (monitored by TLC). The reaction completes after 35 minutes (min), and the solvent is removed under reduced pressure using a rotary evaporator to give a crude product. Petroleum ether and ethyl acetate are used as an eluent for the purification of the crude product via column chromatography on silica gel. A volume ratio of the petroleum ether: the ethyl acetate is 3:1 used herein. And then a target product 48 is rapidly collected after concentration in vacuum, in a yield of 75%. The reaction is expressed by the following formula:

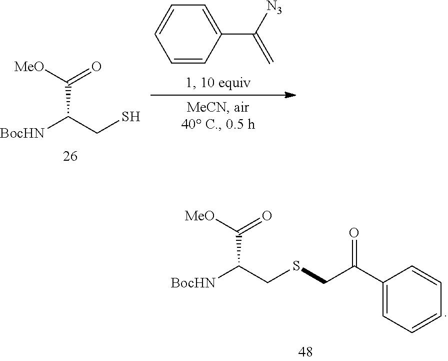

### Embodiment 3

A cysteine derivative 26 (0.4 mmol, 2 equiv) is dissolved in 3 mL of THF to obtain a solution. To this solution, vinyl azide 16 (0.2 mmol, 1 equiv) is added to obtain a reaction mixture. The mixture is stirred under an air atmosphere at 0° C. of ice bath for reacting until peptide in the mixture is consumed (monitored by TLC). The reaction completes after 24 h, and the solvent is removed under reduced pressure using a rotary evaporator to give a crude product. Petroleum ether and ethyl acetate are used as an eluent for the purification of the crude product via column chromatography on silica gel. A volume ratio of the petroleum ether: the ethyl acetate is 3:1 used herein, and then a target product 79 (also referred to a modified product) is collected after concentration in vacuum, in a yield of 99%. The reaction is expressed by the following formula:

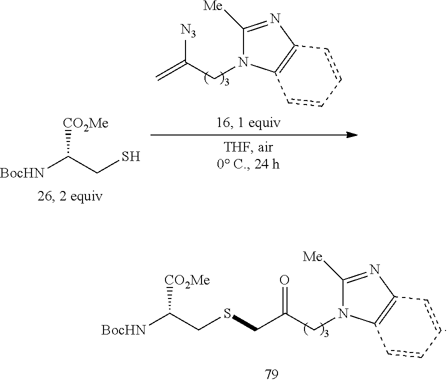

### Embodiment 4

A glutathione 44 abbreviated GSH (0.2 mmol, 1 equiv) is dissolved in 2 mL of phosphate buffer saline abbreviated as PBS (pH 7.4) to obtain a solution. To this solution, vinyl azide 1 (0.4 mmol, 2 equiv) dissolved in 2 mL of THF is added to obtain a reaction mixture. The mixture is stirred at 40° C. under an air atmosphere for reacting until peptide in the mixture is consumed (monitored by TLC, with a solvent mixture of n-butanol:acetic acid:water in a volume ratio of 3:1:1 as the mobile phase). The reaction completes after 4 h, and the solvent is removed under vacuum to obtain a crude product. The crude product is purified by reverse-phase column chromatography with 3% methanol aqueous solution as an elution mobile phase, and a modified product 80 is obtained by lyophilization in a yield of 99%. The reaction is expressed by the following formula:

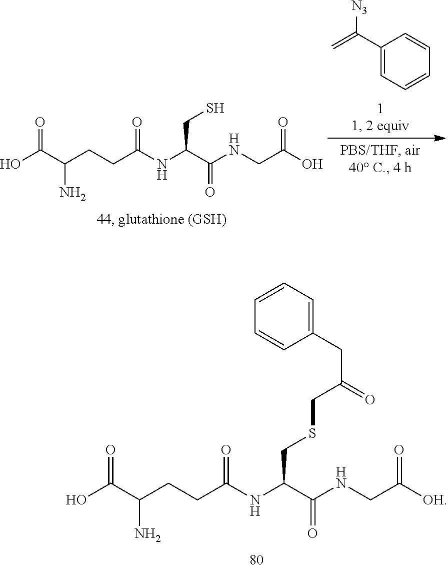

As shown in Table 4, other embodiments are carried out the reaction using the same procedures with the embodiment 4 under other conditions by replacing different solvents. In the Table 4, “a” represents reaction implementation conditions including that temperature and raw materials are the same as those for embodiment 4, “b” represents a separated yield of a target product after the reaction is completed, and “c” and “d” represent pH values of PBS being 7.4 and 7.2 respectively. The conditions “a” in the table 4 represent that the reaction is carried out at 40° C., in which 0.2 mmol of glutathione 44 (1.0 equiv) and 0.4 mmol of vinyl azide 1 (2.0 equiv) are used as raw materials for the reaction. 2.0 mL of the PBS is used as a cosolvent. And the pH value of the PBS is 7.4 under the condition “c”; and the pH value of the PBS is 7.2 under the condition “d”. In addition, the separated yield of the target products in the embodiments of Table 4 is at a range of 73% to 99%. Therefore, it can be seen that the reaction can be performed under various solvents described above.

TABLE 4
Reaction implementation conditions ^{a}

| Serial number | Solvent | Reaction time | | Yield ^{b} |
|---|---|---|---|---|
| 1^{c} | PBS/THF | 4 | h | 99% |
| | (1/1, V/V) | | | |
| 2^{c} | PBS/MeCN | 10 | min | 73% |
| | (1/1, V/V) | | | |
| 3^{c} | PBS/EtOH | 7 | h | 97% |
| | (1/1, V/V) | | | |
| 4^{c} | PBS/DMSO | 48 | h | 98% |
| | (1/1, V/V) | | | |
| 5^{c} | PBS/Dioxane | 12 | h | 99% |
| | (1/1, V/V) | | | |
| 6^{c} | PBS/IPA | 8 | h | 93% |
| | (1/1, V/V) | | | |
| 7^{d} | PBS/THF | 12 | h | 90% |
| | (1/1, V/V) | | | |

### Embodiment 5

A glutathione 44 (0.2 mmol, 1 equiv) is dissolved in 2 mL of PBS (pH 7.4) to obtain a solution. To this solution, vinyl azide 15 (0.4 mmol, 2 equiv) dissolved in 2 mL of THF is added to obtain a reaction mixture. The mixture is stirred at 30° C. under an air atmosphere for reacting until peptide in the mixture is consumed (monitored by TLC using a solvent mixture of n-butanol: acetic acid: water in a volume ratio of 3:1:1 as the mobile phase). The reaction completes after 48 h, and the solvent is removed under reduced pressure using a rotary evaporator to obtain a crude product. The crude product is purified by reverse-phase column chromatography by using 3% methanol aqueous solution as an elution mobile phase, and a modified product 85 is collected by lyophilization in vacuum, in a yield of 78%. The reaction is expressed by the following formula:

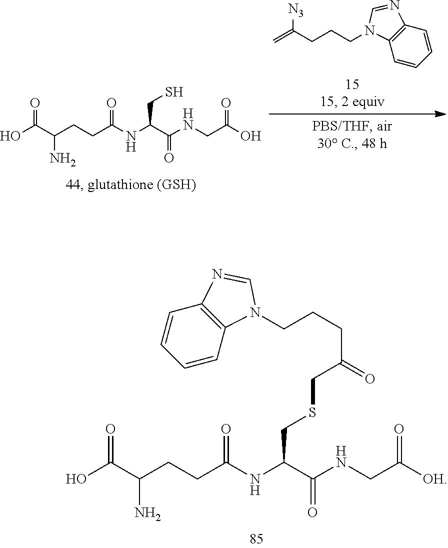

### Embodiment 6

A glutathione 44 (0.2 mmol, 1 equiv) is dissolved in 2 mL of PBS (10 millmoles per liter abbreviated as mmol/L, pH 7.4) to obtain a solution. To this solution, vinyl azide 18 (0.4 mmol, 2 equiv) dissolved in THF is added to obtain a reaction mixture. The mixture is stirred at 60° C. under an air atmosphere for 8 h, and the reaction is analyzed by high performance liquid chromatography (HPLC-MS). Thereafter, a target product 68 is collected in a yield of 63%. The reaction is expressed by the following formula:

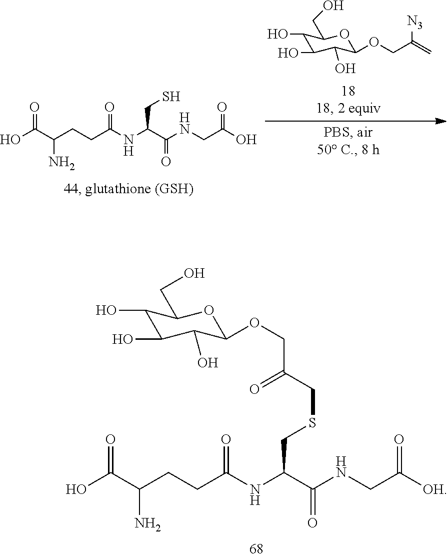

### Embodiment 7

A glutathione 44 (0.2 mmol, 1 equiv) is dissolved in 2 mL of PBS (10 mmol/L, pH 7.4) to obtain a solution. To this solution, vinyl azide 22 (0.4 mmol, 2 equiv) is added to obtain a reaction mixture. The mixture is stirred at 40° C. under an air atmosphere for 10 min, and the reaction is analyzed by HPLC-MS. Thereafter, a target product 77 is collected in a yield of 81%. The reaction is expressed by the following formula:

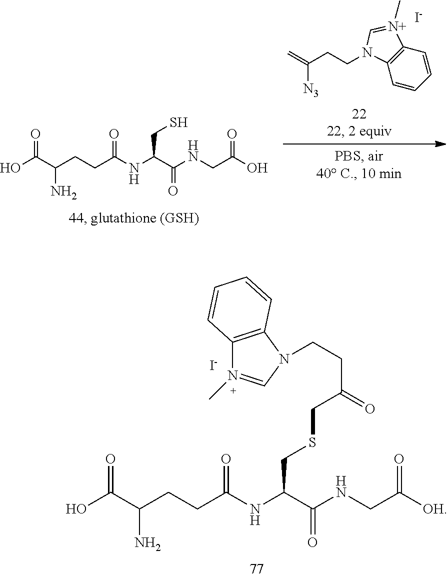

### Embodiment 8

A reaction in the embodiment 8 is expressed by the following formula:

The above reaction formula illustrates that a dansyl-vinyl azide is combined with a bovine serum albumin 46 (BSA).

A modification method for BSA 46 is performed as follows. The reaction is carried out in a 1.5 mL centrifuge tube, 10 microliter (μL) PBS solution of BSA 46 (1.5×10⁻⁶ mmol, 1 equiv), 40 μL ethylalcohol (EtOH) and 140 μL PBS (10 mmol/L, pH 7.4) (in another embodiment with 90 μL EtOH and 90 μL PBS) are added to the 1.5 mL centrifuge tube to obtain a premixed solution. 10 μL of phenylalkenyl azide 6 dissolved in EtOH (1.5×10⁻⁴ mmol, 100 equiv) is added to the premixed solution to obtain a protein solution with a final volume of 200 μL and a protein content of 1.5×10⁻⁶ mmol. Then, the centrifuge tube is sealed with a preservative film and is punctured with a toothpick, followed by shaking at 40° C. for 24 h to collect a target product 88 (also referred to as a sample), and the target product 88 is analyzed by Sodium dodecyl sulphate polyacrylamide gel electrophoresis (SDS-PAGE, also referred to as protein electrophoresis analysis).

The protein solution is mixed with SDS loading buffer to obtain a sample, and 100 mL of 10% separation gel (consisting of 0.25 mole per liter abbreviated as M of tris-(hydroxymethyl)-aminomethane hydrochloride abbreviated as Tris-HCl, 10% of SDS, 30% of glycerol and 0.05% of bromophenol blue) is first prepared. After the separation gel is solidified, 100 mL of SDS-PAGE concentrated gel (consisting of 15% acrylamide, 0.375 M of Tris with a pH value of 8.8, 0.1% SDS, 0.1% ammonium persulfate abbreviated as APS and 0.05% N,N,N′,N′-tetramethylethylenediamine abbreviated as TEMED) is added to the solidified separation gel to make the gel fully solidified. Then, the sample and PageRuler™ Plus Prestained Protein Marker are loaded into loading wells respectively to run in a buffer (25 millmoles per liter abbreviated as mM of Tris, 0.19 M of glycine and 0.1% of SDS). The fluorescence of protein in the obtained gel is captured and recorded with Gel Doc™ XR+gel imager and Image lab™ software, and the gel is recorded again with the gel imager after sample staining on 10 μL of Coomassie blue dye (CBB) R250 with a concentration of 2.5 grams per liter (g/L).

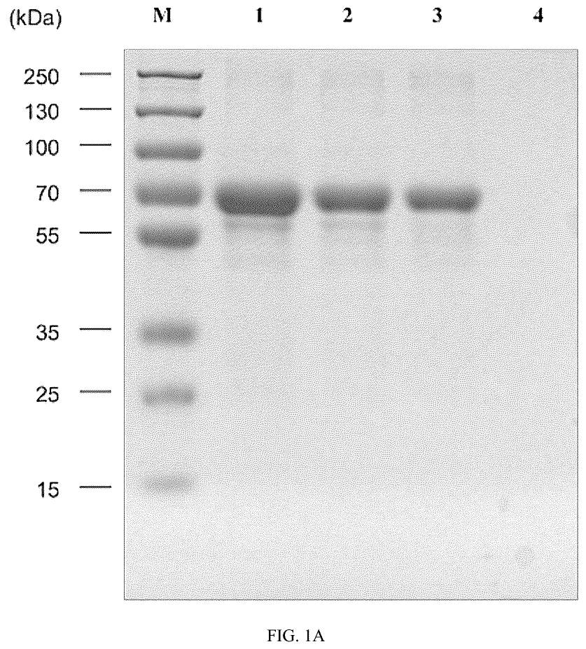

As shown in FIGS. 1A-1B, M represents the PageRuler™ Plus Prestained Protein marker. Lane 1 represents a result of the reaction under a condition of a volume ratio of EtOH: PBS being 1:1 (i.e., V/V=1:1), and the obtained BSA-dansyl conjugate 88 is treated with a 5×SDS-PAGE sample buffer and boiled at 100° C. for 5 min. Lane 2 represents a result of the reaction under a condition of a volume ratio of EtOH: PBS being 1:3 (i.e., V/V=1:3), and the obtained BSA-dansyl conjugate 88 is treated with a 5×SDS-PAGE sample buffer and boiled at 100° C. for 5 min. Lane 3 represents a result of an unmodified BSA. Lane 4 represents a result of a dansyl-vinyl azide 6.

As shown in FIGS. 1A-1B, FIG. 1A illustrates a CBB staining electrophoresis diagram, and FIG. 1B illustrates a fluorescence diagram. Compared FIG. 1A and FIG. 1B, the BSAs modified by the dansyl-vinyl azide 6 show the fluorescence as shown in lanes 1-2 of FIG. 1B. However, only the BSA in the lane 3 has no fluorescence phenomenon, indicating that the BSA is successfully modified by the dansyl-vinyl azide 6 to obtain a BSA-dansyl conjugate. Therefore, fluorescence marking of BSA is completed.

### Embodiment 9

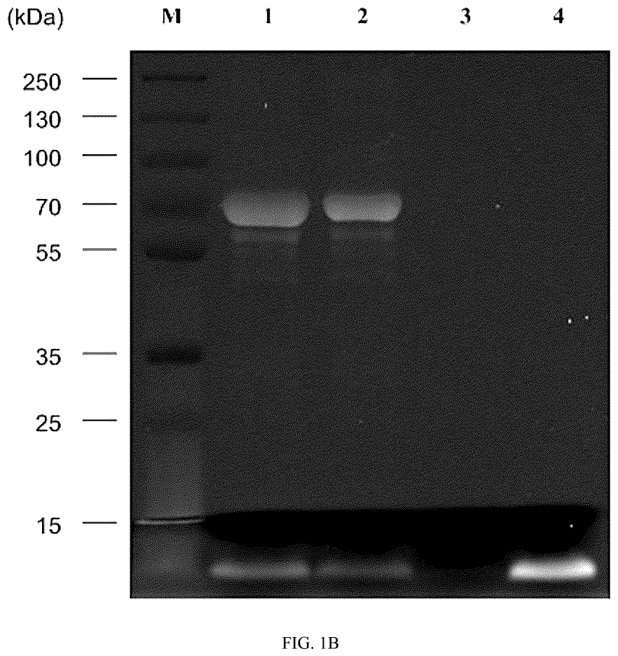

A ternary fluorescent protein conjugate prepared by labeling Ni-NTA resin with YPet-ECFP (referred to as a combination of modified yellow fluorescent protein with enhanced cyan fluorescent protein) and STAV AF568 (referred to as a dye for the streptavidin Alexa Fluor™ 568 conjugate), and a preparation method of the ternary fluorescent protein conjugate is shown in FIG. 2.

A biotin 7 is dissolved in THF and diluted to 8.4×10⁻³ micromoles per microliter (μmol/μL); a streptavidin Alexa Fluor™ 568 conjugate is dissolved in PBS (pH 7.4) and diluted to 36 micromoles per liter (μM), and the concentration of the purified YPet-ECFP 47 protein is determined to be 0.5808 micrograms per microliter (μg/μL) by a BCA Protein Assay kit (referred to as a determination kit of bicinchoninic acid for protein).

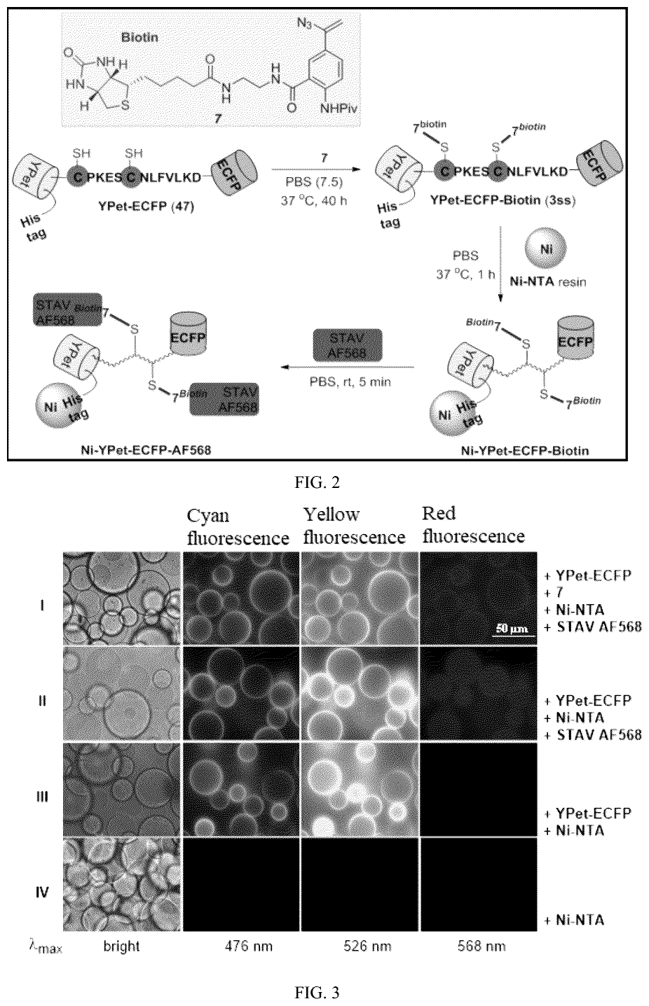

The reaction is carried out in two 500 μL centrifuge tubes including a first centrifuge tube and a second centrifuge tube. The biotin 7 (10 μL, 8.4×10⁻² μmol, 100 equiv) and the YPet-ECFP 47 (100 μL, 8.4×10⁻⁴ μmol, 1 equiv) are added to the first centrifuge tube, and the YPet-ECFP 47 (100 μL, 8.4×10⁻⁴ μmol, 1 equiv) is added to the second centrifuge tube. The two centrifuge tubes added with reaction mixtures are gently shaken at 37° C. for about 40 h. Then, 50 μL of nickel-nitrilotriacetic acid (NTA) agarose beads (Ni-NTA) (commercially available from Thermo Scientific) are added to the two reaction mixtures individually and shaken at room temperature for 1 h to ensure that the YPet-ECFP in the two individual centrifuge tubes is completely combined with the Ni-NTA resin. The Ni-NTA resin and its adsorbate are subjected to centrifugal precipitation in a low-speed centrifuge, and supernatant is removed. 1 mL of PBS is used to rinse each of the two centrifuge tubes for three times to rinse away excess biotin 7, and the streptavidin Alexa Fluor™ 568 (12.5 μL, 4.5×10⁻⁴ μmol, 36 μM) and PBS (100 μL) are added to the two centrifuge tubes individually, followed by shaking at room temperature for 5 min, rinsing the two reaction mixtures with 1 mL of PBS for three times to remove excess streptavidin Alexa Fluor™ 568, finally adding 100 μL of PBS to suspend the Ni-NTA resin, and placing the two reaction mixtures at a bottom of a confocal disk for microscope imaging (a type of the microscope of Laril Eiss, Germany, Primo Vert). According to imaging results of the microscope, the Ni beads do not have fluorescence (as shown in part IV of FIG. 3), and it can be determined that the presented fluorescence is the fluorescence of the adsorbate of the Ni-NTA resin. Cyan fluorescence and yellow fluorescence shown in parts I, II, III of FIG. 3 are mainly derived from YPet-ECFP 47. The red fluorescence in parts I and II of FIG. 3 is derived from the STAV AF 568 dye in the streptavidin Alexa Fluor™ 568 conjugate. When the azide is reacted with the sulfhydryl group contained in the cysteine on the protein, the biotin on the azide can be adsorbed on the surfaces of the Ni beads along with the protein. When the STAV AF 568 dye is added, due to a specific binding of biotin to avidin, the dye is preferentially attached to the protein by biotin-avidin interaction and adsorbed on the surfaces of the Ni beads. Therefore, a layer of red light ring on the surfaces of the Ni beads can be observed, the position thereof is consistent with that of cyan fluorescence and yellow fluorescence (as shown in part I of FIG. 3). Therefore, the red fluorescence can be obtained, and the red fluorescence is presented by adsorbing the Ni beads through the protein. If the azide modification reaction does not occur, after the dye is added, the dye can evenly permeate into the Ni beads from the outside (as shown in part II of FIG. 3). In both cases of parts I and II of FIG. 3, the optical density around the Ni beads is treated and analyzed, it is found that the light density around the modified Ni beads is greater than that of the unmodified Ni beads, although there is with a difference on statistics. Therefore, the red fluorescence in the streptavidin Alexa Fluor™ 568 conjugate is obtained by a structure of biotin-azide-sulfhydryl group attached to the surfaces of the Ni beads, proving that the azide modification reaction can also occur quickly on the protein level.

What is claimed is:

1. A polypeptide or protein directional modification method based on sulfhydryl-alkenyl azide coupling, comprising:
reacting a sulfhydryl group-containing compound with a compound containing alkenyl azide group in a reaction medium to modify the sulfhydryl group-containing compound into a compound containing β-carbonyl sulfide;
wherein a reaction temperature is in a range of 0 degrees Celsius (° C.) to 60° C. and a reaction time is in a range of 10 minutes (min) to 48 hours (h); and
wherein the sulfhydryl group-containing compound is one selected from the group consisting of a cysteine, a cysteine derivative, a sulfhydryl group-containing polypeptide, and a sulfhydryl group-containing protein.

2. The polypeptide or protein directional modification method based on sulfhydryl-alkenyl azide coupling according to claim 1, wherein a general structural formula of the cysteine and the cysteine derivative is expressed by a formula (I) as follows: 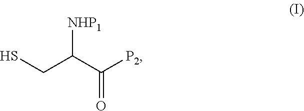
where P₁ in the formula (I) represents one selected from the group consisting of a hydrogen group, an alkyl group, an aryl group, a heteroaryl group, a carbonyl-alkyl group, a carbonyl-aryl group, a carbonyl-heteroaryl group, an ester-alkyl group, an ester-aryl group, an ester-heteroaryl group, a carbonyl-amino-alkyl group, a carbonyl-amino-aryl group, a carbonyl-amino-heteroaryl group, a dipeptidyl group and a polypeptidyl group; and
where P₂ in the formula (I) represents one selected from the group consisting of a hydroxyl group, an ether-alkyl group, an amino-alkyl group, an amino-aryl group, an amino-heteroaryl group, a cyclic amino group, an amino-dipeptidyl group and an amino-polypeptidyl group.

3. The polypeptide or protein directional modification method based on sulfhydryl-alkenyl azide coupling according to claim 1, wherein a general structural formula of the compound containing an alkenyl azide group is expressed by a formula (II) as follows: 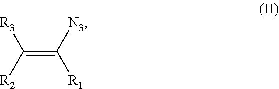
where each of R₁, R₂ and R₃ in the formula (II) represents one selected from the group consisting of a hydrogen group, an alkyl group, an aryl group, a heteroaryl group, an alkynyl group, an alkenyl group.

4. The polypeptide or protein directional modification method based on sulfhydryl-alkenyl azide coupling according to claim 3, wherein R₁ in the formula (II) of the compound containing an alkenyl azide group is substituted by at least one Q¹ group; and
wherein the Q¹ group is one selected from the group consisting of a hydrogen group, a halogen group, a hydroxyl group, an amino group, a cyano group (—CN), a trifluoromethyl group (—CF₃), a trifluoromethoxy group (—O—CF₃), a nitro group (—NO₂), an azide group, an alkyl group, an alkenyl group, an alkynyl group, a cycloalkyl group, a heterocycloalkyl group, an aryl group, a heteroaryl group, a heterocyclic aryl group, an —OR₄ group, a —S(O)ₙR₅ group, a —NR₆R₇ group, a —SO₂NR₆R₇ group, a carbonyl-R₅ group, a carbonyl-NR₆R₇ group, an ester-R₅ group, and an ester-R₅ group.

5. The polypeptide or protein directional modification method based on sulfhydryl-alkenyl azide coupling according to claim 1, wherein the compound containing β-carbonyl sulfide is one selected from the group consisting of a cysteine containing β-carbonyl sulfide, a cysteine derivative containing β-carbonyl sulfide, a polypeptide containing β-carbonyl sulfide, and a protein containing β-carbonyl sulfide.

6. The polypeptide or protein directional modification method based on sulfhydryl-alkenyl azide coupling according to claim 5, wherein a general structural formula of the cysteine containing β-carbonyl sulfide and the cysteine derivative containing β-carbonyl sulfide is expressed by a formula (III) as follows: 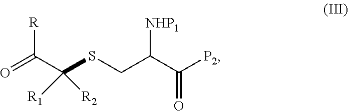
where P₁ in the formula (III) represents one selected from the group consisting of a hydrogen group, an alkyl group, an aryl group, a heteroaryl group, a carbonyl-alkyl group, a carbonyl-aryl group, a carbonyl-heteroaryl group, an ester-alkyl group, a ester-aryl group, an ester-heteroaryl group, a carbonyl-amino-alkyl group, a carbonyl-amino-aryl group, a carbonyl-amino-heteroaryl group, a dipeptidyl group, and a polypeptidyl group;
where P₂ in the formula (III) represents one selected from the group consisting of a hydroxyl group, an ether-alkyl group, an amino-alkyl group, an amino-aryl group, an amino-heteroaryl group, a cyclic amine group, an amino-dipeptidyl group and an amino-polypeptidyl group; and
where R₁ in the formula (III) represents one of an alkyl group and an aryl group, R₂ and R₃ in the formula (III) each represent a hydrogen group.

7. The polypeptide or protein directional modification method based on sulfhydryl-alkenyl azide coupling according to claim 1, wherein the reaction medium is at least one selected from the group consisting of tetrahydrofuran, dioxane, acetone, N, N-dimethylformamide, N-methyl pyrrolidone, dimethyl sulfoxide, water, methanol, ethanol, isopropanol, acetonitrile, and a buffer solution.

8. The polypeptide or protein directional modification method based on sulfhydryl-alkenyl azide coupling according to claim 1, wherein the sulfhydryl group-containing compound is one of the group consisting of the followings: 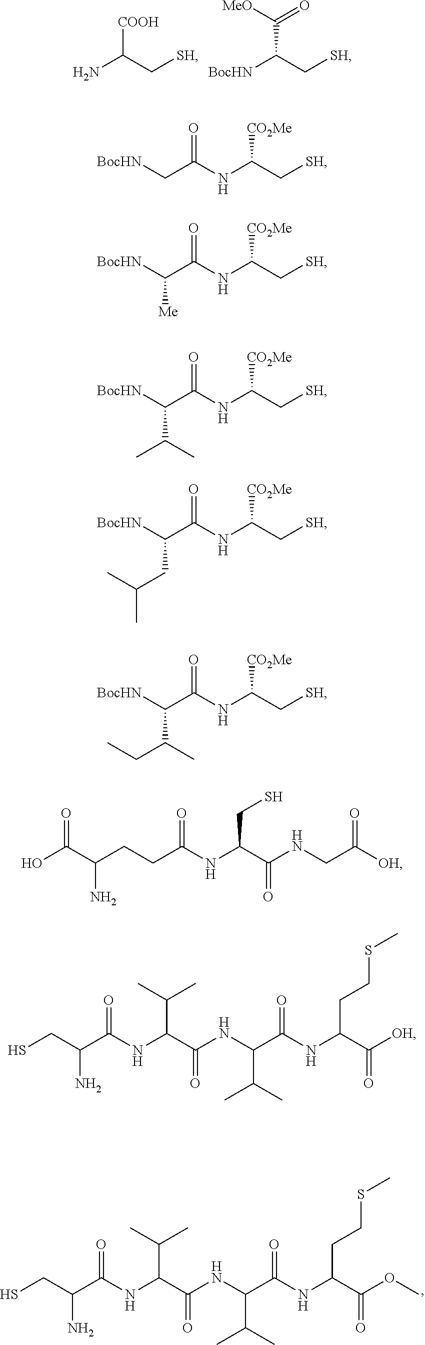 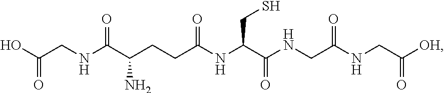 [SMILES: N[C@@H](CCC(=O)N[C@@H](CS)C(=O)NCC(=O)NCC(=O)O)C(=O)NCC(=O)O]
a bovine serum albumin (BSA), and a modified protein.

9. The polypeptide or protein directional modification method based on sulfhydryl-alkenyl azide coupling according to claim 1, wherein the compound containing an alkenyl azide group is one of the group consisting of the followings: 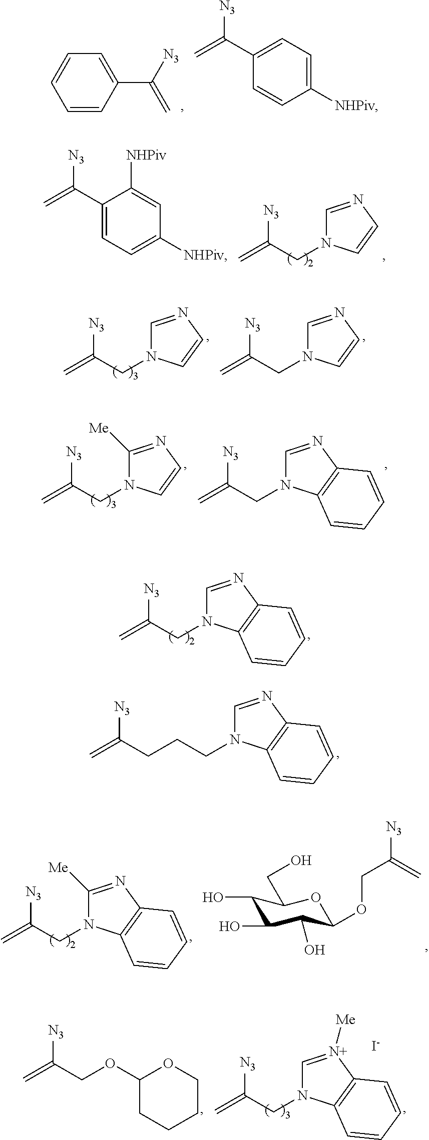
a dansyl-vinyl azide and a streptavidin Alexa Fluor™ 568 conjugate.

10. The polypeptide or protein directional modification method based on sulfhydryl-alkenyl azide coupling according to claim 4, wherein R₄, R ₅, R₆ and R₇ in the Q¹ group is substituted by one selected from the group consisting of at least one hydrogen group, a halogen group, a cyano group (—CN), a hydroxyl group (—OH), an amino group (—NH₂), a nitro group (—NO₂), an oxyl group, a trifluoromethyl group (—CF₃), a trifluoromethoxy group (—O—CF₃), a carboxyl group (—COOH), a —S(O)ₙH group, an alkyl group, an aryl group, a heteroaryl group, a cycloalkyl group, a heterocycloalkyl group, a heterocycloaryl group, and an ether-alkyl group; where n is taken from 0, 1, or 2.

11. The polypeptide or protein directional modification method based on sulfhydryl-alkenyl azide coupling according to claim 1, wherein the reaction temperature is in a range of 37° C. to 40° C.
